(19) United States
(12) Patent Application Publication
(10) Pub. No.: US 2019/0053856 A1
(43) Pub. Date: Feb. 21, 2019

(54) METHOD AND SYSTEM FOR POSE CONTROLLED ABLATION

(71) Applicant: UNIVERSITAT BERN, Bern (CH)

(72) Inventors: Stefan WEBER, Boll (CH); Daniel CANDINAS, Bern (CH); Pascale TINGUELY, Bern (CH)

(73) Assignee: UNIVERSITAT BERN, Bern (CH)

(21) Appl. No.: 16/068,671

(22) PCT Filed: Jan. 7, 2017
(86) PCT No.: PCT/EP2017/050290

§ 371 Date: Jul. 8, 2018

(30) Foreign Application Priority Data
Jan. 7, 2016 (EP) 16150502.9

(51) Int. Cl. A61B 34/20; A61B 18/04

(52) U.S. Cl. CPC A61B 34/20; A61B 2018/00577; A61B 18/04

(57) ABSTRACT

The present invention relates to a system (S) for creating an ablation volume (20) within a target object (1), comprising: an applicator (2) comprising a head (2a), which applicator (2) is configured to be pinserted into the patient's body so as to position said head (2a) into or close to a target object (1) located inside said body, and wherein the applicator (2) is configured to emit energy (E) provided to the applicator (2) via said head (2a) so as to create an ablation volume (20) comprising at least a region of said target object (1), a tracking system (6) configured to track the pose (4) of the head (2a) with respect to the pose (5) of the target object (1), wherein the tracking system (6) is further configured to provide a signal (6a) indicative of the relative pose of the head (2a) of the applicator (2) with respect to the target object (1), and an energy control unit (10) configured to control emission of said energy (E) by the head (2a) of the applicator (2) in response to said signal (6a).

The invention relates to a system for creating an ablation volume as well as a method for controlling the amount of energy emitted per time by a head of an applicator for creating an ablation volume within a target object.

Ablation refers to the medical process of indirect removal of organs and/or parts thereof by destruction through introduction of any type of energy into the body. More specifically, in oncology tumors are destroyed by introducing energy (i.e. heat) or cold into the tumor tissue, causing the tumor tissue to be destroyed. To date, medical ablations are typically carried out using needle like applicators that are manually or robotically placed in or near an anatomical target and where the placement process is supported by medical imaging and if available instrument guidance technology.

Based on the above the problem underlying the present invention is to provide a system for creating an ablation volume that allows a more precise and reproducible creation of an ablation volume with respect to the target object.

This problem is solved by a system having the features of claim 1. Preferred embodiments are stated in the sub claims and are described below.

According to claim 1 said system for creating an ablation volume comprising a target object or at least a portion thereof, comprises:
an applicator comprising a head, which applicator is configured to be inserted into the patient's body so as to position said head into or close to a target object (e.g. a tumor) located inside said body, and wherein the applicator is configured to emit via said head energy provided to the applicator so as to create an ablation volume comprising at least a region of said target object,
a tracking system configured to track the pose of the head with respect to the pose of the target object, wherein the tracking system is further configured to provide a signal indicative of the relative pose of the head of the applicator with respect to the target object, and
an energy control unit configured to control emission of said energy by the applicator in response to said signal.

Particularly, in the framework of the present invention, the notion pose refers to the position and the orientation of the respective body and therefore particularly comprises at least six parameters, namely three parameters for characterizing the position of the respective body, and three parameters for characterizing the orientation of the respective body.

Preferably, said applicator is formed as an elongated needle to allow for a percutaneous access to the body of the patient.

Using the system according to the present invention, ablation volumes can be created that are dependent in their properties (e.g. position, orientation, size and shape) from the spatial relationships between the applicator head and the target object.

This allows to control the correct creation of ablation volumes with respect to a target structure with better precision. Further, irregular shaped ablation volumes might be created that allow the complete ablation of irregular shaped target objects with the best available sensitivity and specificity.

According to a preferred embodiment of the present invention, the energy control unit is configured to automatically trigger emission of energy via the head of said applicator towards the target object for generating said ablation volume when the distance between the head and the target object as measured by the tracking system falls below a pre-defined value.

Further, according to a preferred embodiment of the present invention, the tracking system is configured to determine the pose of the head of the applicator and the pose of the target object using at least one of: electromagnetic tracking, optical tracking, optical-fiber based tracking, time-of-flight-based tracking, or any combination thereof.

Particularly, in electromagnetic tracking, a transmitter (magnetic field generator) is preferably used to induce a current in sensor coils that can be embedded into the tracked objects. Further, in optical tracking, a stereo camera is preferably used to track fiducial markers that are attached to the instrument or anatomical structure of interest. Furthermore, in optical-fiber-based tracking, the bending of a fiber optics is measured through fibre bragg gates (FBG) to estimate the location and orientation along its length. Finally, in time-of-flight based tracking, a range imaging camera system may be used that resolves distance based on the known speed of light, measuring the time-of-flight of a light signal between the camera and the subject for each point of the image.

Further, in an embodiment of the present invention, the tracking means/system comprise(s) a display means (e.g. a display for displaying information optically) for displaying the position or pose of the head of the applicator with respect to the position or pose of the target object, preferably in real-time.

Further, according to a preferred embodiment of the present invention, the energy control unit is configured to dynamically control the temporal and spatial delivery of energy to the target object via the head of the applicator using said signal which is particularly provided by the tracking system in a quasi-continuous fashion. Thus the energy control unit controls the emission of energy depending on said relative pose of the head of the applicator with respect to the target. Further, temporal control particularly means that the emission of energy is a distinct function of time.

Further, according to an embodiment, the energy control unit is configured to control emission of energy, such that the energy that is delivered per unit time and unit of volume to the target object is a linear or non-linear (particularly continuous) function derived from the relative pose (i.e. 6D) between the head and the target object and/or from a derivative of said relative pose (i.e. a speed, an acceleration etc.).

Particularly, said delivered energy may increase and/or decrease depending on the distance between the head and the target object.

Particularly, said delivered energy may increase and/or decrease depending on the position of the head with respect to the target object.

Further, particularly, said delivered energy may increase and/or decrease depending on the position of the head and on the spatial orientation of the applicator or of the head with respect to the target object (i.e. depending on the relative pose).

Further, according to a preferred embodiment of the present invention, said energy which the applicator is configured to emit via its head causes the target object to heat up within said ablation volume so that subsequently material properties of the target object in the ablation volume are changed. Particularly, said energy is one of: energy in the form of alternating electromagnetic fields (i.e. radiofrequency, microwaves, light) or other sorts of energy.

Further, according to a preferred embodiment of the present invention, the applicator comprises a handle for manually moving the applicator or a means for automatically moving the applicator. Using a means for moving the applicator automatically eases creation of an arbitrarily shaped ablation volume, particularly according to a preexisting image-based plan.

Further, according to a preferred embodiment of the present invention, the system is configured to generate an ablation volume of a pre-defined shape by moving the applicator along a pre-defined track by means of the means for automatically moving the applicator.

Further, according to a preferred embodiment of the present invention, the tracking system comprises a first tracking device for measuring the pose of the target object, wherein said first tracking device is configured to be placed into the body of a patient comprising said target object inside said body, and wherein preferably the first tracking device is a first transmitter configured for transmitting an electromagnetic signal indicative of the position or pose of the target object. However, alternatively said first tracking device is an externally attached first tracking device, i.e., is arranged outside the patient.

Further, according to a preferred embodiment of the present invention, the tracking system comprises a second tracking device for measuring the pose of the applicator head, wherein said second tracking device is arranged on the applicator, particularly on the head of the applicator, and wherein preferably the second tracking device is a second transmitter configured for transmitting an electromagnetic signal indicative of the position or pose of the head of the applicator.

Further, according to a preferred embodiment of the present invention, the system comprises an elongated flexible means (or an elongated flexible device) comprising said first tracking device at a tip of said flexible means (or device), wherein said flexible means (or device) is configured to be placed into or close to the target object via a body lumen of the patient, particularly via the arterial or venous vascular system of the patient.

Preferably, according to a preferred embodiment of the present invention, said elongated flexible means or device is a intravascular catheter, preferably configured for transcatheter arterial chemoembolization (also denoted as TACE), or a guide wire for a catheter (e.g. of a catheter configured for TACE).

Here, particularly said catheter or guide wire comprising said first tracking device in the form of an electromagnetic transmitter is guided as close as possible to the target object (e.g. a tumor). Using a subtraction angiography, the pose of the target object with respect to the first tracking device can be inferred and a spatial target position can be defined. The head of the applicator comprises the second tracking device which also is an electromagnetic transmitter. The relative position or pose of the head of the applicator with respect to the guide wire or catheter (second tracking device) is continuously measured and may be displayed on a display device. The user can thus guide the applicator head precisely to the target object.

Further, according to a preferred embodiment of the present invention, the applicator head is configured to provide directionality of energy distribution, wherein the energy control unit is particularly configured to control the resulting spatial energy distribution in the target object as a function of at least one of: the relative pose of the head of the applicator with respect to the target object, the orientation of the head with respect to the target object, the position of the head with respect to the target object, medical image data, a property of the target object.

Furthermore, according to an embodiment, the head of the applicator comprises a plurality of independent energy emitting elements for delivering energy to the target object, wherein said elements are particularly arranged side by side along a longitudinal axis of the head and/or side by side along a periphery of the head (which periphery extends along a plane that runs perpendicular to said longitudinal axis). Particularly, the energy control unit is configured to control the respective energy emitting element so as to generate said resulting spatial energy distribution.

Further, according to a preferred embodiment of the present invention, the system comprises a plurality of applicators, wherein each applicator comprises a head and is configured to be inserted into the patient's body so as to position the respective head into or close to said target object (particularly a tumor) located inside said body, and wherein the respective applicator is configured to emit energy provided to the respective applicator via the respective head so as to create an ablation volume having a pre-defined shape and comprising at least a region of said target object.

Furthermore, yet another aspect of the present invention relates to a method for controlling the amount of energy emitted per time by a head of an applicator for creating an ablation volume within a target object as claimed in claim 20.

According thereto, data relating to the relative pose of the head of the applicator with respect to the pose of the target object is measured by means of a tracking system upon guiding the applicator to the target object, and wherein the energy emitted by said head of the applicator is automatically controlled depending on said relative pose of the applicator with respect to the target object.

Further, according to a preferred embodiment of the present invention, both the pose of applicator's head and the pose of the target are measured by means of one of: electromagnetic, optical, optical-fiber-based or time-of-flight based tracking (see also above).

Further, according to a preferred embodiment of the present invention, the temporal and spatial delivery of energy via the head of the applicator is dynamically controlled based on an quasi-continuous input from the tracking system.

Further, according to a preferred embodiment of the present invention, energy (preferably in the form of alternating electromagnetic fields, e.g. radiofrequency, microwaves, light or other sorts of energy) is emitted via the applicator's head into the target object causing a tissue volume of interest near or around the target object or comprising the target object or at least portions thereof to be destroyed.

Further, according to a preferred embodiment of the present invention, the head of the applicator is guided manually to the target object.

Further, according to a preferred alternative embodiment of the present invention, the head of the applicator is automatically guided to the target object using a means for automatically moving the applicator, e.g. a robotic arm.

Further, according to a preferred embodiment of the present invention, said means is configured to move the head of the applicator upon emitting energy to the target object in a pre-defined way so as to give the ablation volume a pre-defined shape that is difficult or impossible to achieve manually otherwise.

Further, according to a preferred embodiment of the present invention, the position or pose of the target object is measured using an externally attached tracking device or using an internally placed tracking device (see also above), wherein particularly an internally placed tracking device (e.g. a beacon) may be placed using a needle like or catheter-like instrument near or within the target volume to be ablated as described above.

Further, according to a preferred embodiment of the present invention, both the pose of the head of the applicator and the pose of the target object are co-registered to a previously or instantaneously acquired medical image data set, allowing for subsequent annotations of anatomical structures and/or simulation and extrapolation of the ablation effects.

Further, according to a preferred embodiment of the present invention, the head of the applicator provides directionality of energy distribution, wherein particularly the resulting spatial energy distribution in the target object is controlled as a function of at least one of: the relative position of the applicator and the target object, available image data, and additionally available target properties.

Further, according to a preferred embodiment of the present invention, a plurality of applicators that may be designed as the applicator described herein, are used, wherein these applicators are introduced synchronously or asynchronously into or near the target object.

Further, according to a preferred embodiment of the present invention, energy is emitted into the target object via said plurality of individual applicators (e.g. via the respective head) so as to create a defined ablation volume according to a plan.

Further, according to a preferred embodiment of the present invention, the creation of the pre-defined ablation volume is automatically controlled according to the pre-defined image-based plan.

Further features, embodiments, aspects and advantages of the present invention shall be described by means of a detailed description with reference to the Figures, wherein

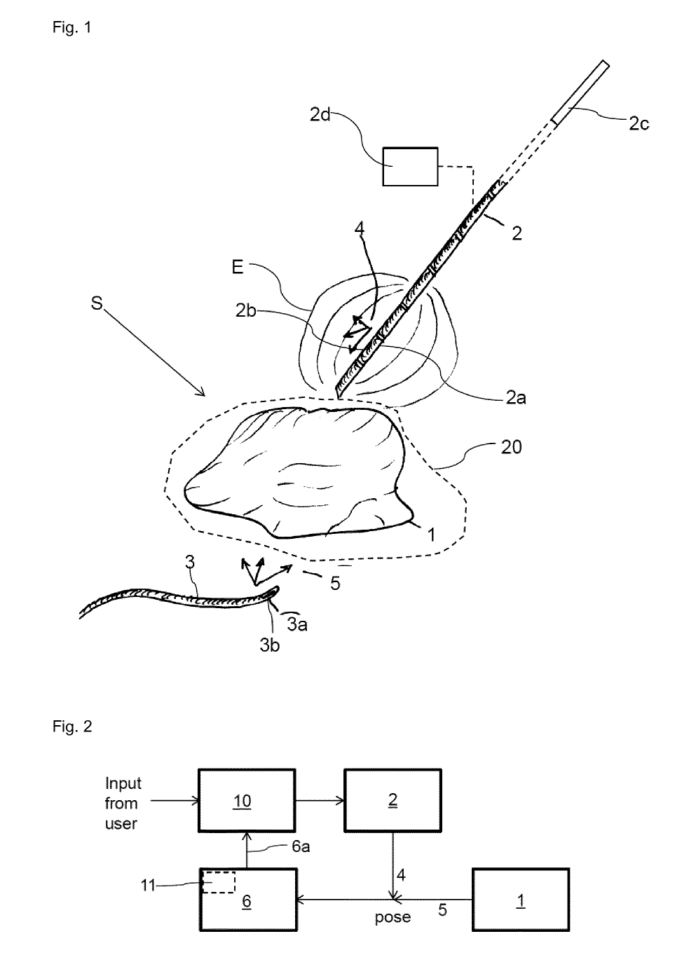

FIG. 1 shows a schematic representation of a system according to the invention;

FIG. 2 shows the communication of individual components of the system according to the invention; and

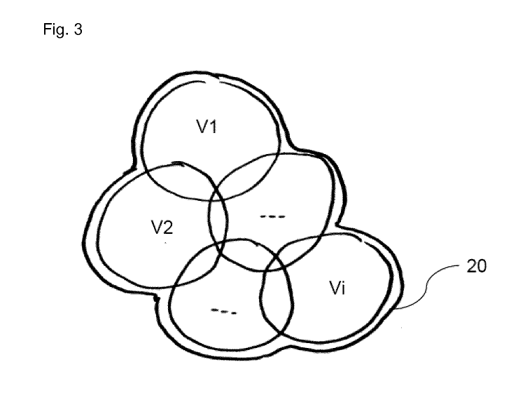

FIG. 3 shows the possibility of a superposition of individual ablation volumes using the system/method according to the invention in order to create an overall ablation volume having a complex, pre-defined shape; and

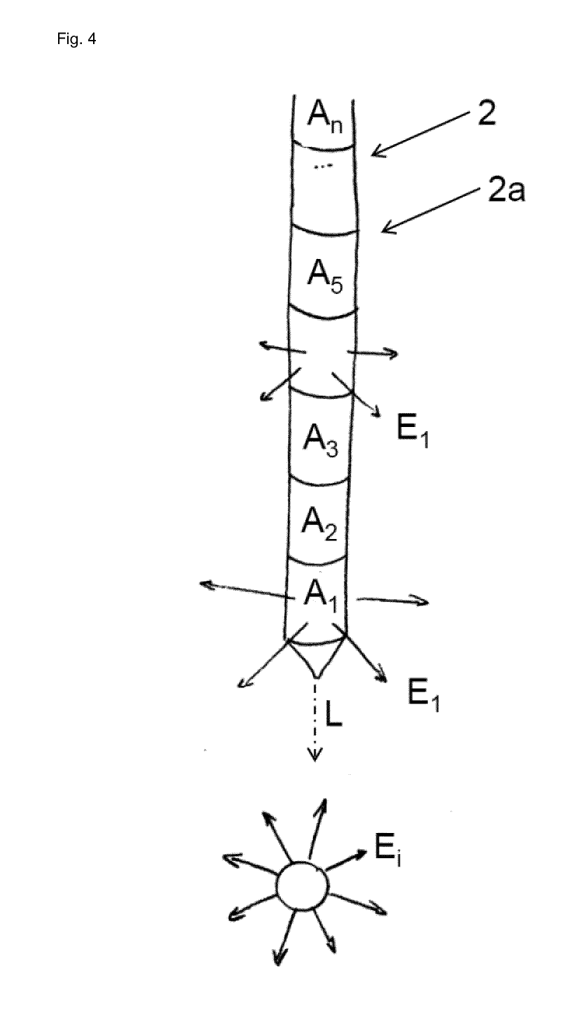

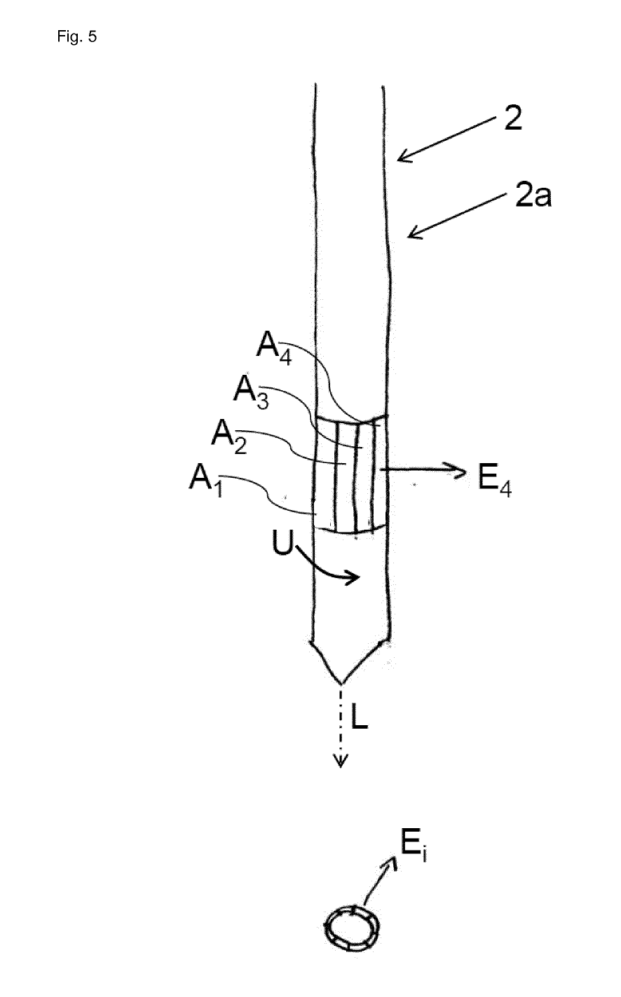

FIGS. 4 to 6 show different heads of an applicator according to the present invention comprising a plurality of energy emitting elements, respectively.

FIG. 1 shows in conjunction with FIGS. 2 and 3 a system S for creating an ablation volume 20 within a target object 1 which may be a tumor located inside a patient's body that shall be destroyed by generating an ablation volume 20 that includes the target object 1, i.e., by heating the target object 1 up in a sufficient manner.

For this, the system S comprises an applicator 2, particularly in the form of an elongated needle that can be percutaneously brought into the vicinity of the target object 1. The applicator comprises a head 2a via which the applicator 2 is configured to emit energy E provided to the applicator 2 by means of a suitable energy source. Such applicators 2 are for instance described in [1].

This allows one to create an ablation volume 20 by controlling the amount of energy E emitted per time via said head 2a towards the target object 1.

The system further comprises a tracking system 6 configured to track the pose 4 of the head 2a with respect to the pose 5 of the target object 1, wherein the tracking system 6 is further configured to provide a signal 6a indicative of the relative pose of the head 2a of the applicator 2 with respect to the target object 1, and an energy control unit 10 configured to control emission of said energy E by the head 2a of the applicator 2 in response to said signal 6a. Particularly the energy control unit 10 is configured to automatically trigger emission of energy E via the head 2a of said applicator 2 towards the target object 1 for generating said ablation volume 20 when the distance between the head 2a and the target object 1 falls below a pre-defined value.

For proving data 6a concerning the spatial position of the head 2a of the applicator 2 with respect to the target object 1, the tracking system 6 may be configured to use a first tracking device 3b for measuring the pose 5 of the target object 1

Here, the system may comprises an elongated flexible means or device 3 (e.g. a catheter) comprising said first tracking device 3b at a tip 3a of said flexible means 3, wherein said flexible means 3 is configured to be placed into or close to the target object 1 via a body lumen of the patient. The first tracking device is preferably configured to transmit an electromagnetic signal which is indicative of the position of the tip 3a of the flexible means 3. Thus, by bringing the tip 3a of the flexible means 3 close to the target object 1, the first tracking device 3b actually reveals the position of the target object 1. A spatial offset between the target object 1 and said tip 3a may be determined by medical image data so that one can precisely determine the position of the target object once the position of the tip 3a is known.

The tracking system 6 may further comprise a second tracking device 2b which may also be formed as a (second) transmitter that is configured to transmit an electromagnet signal as well that is indicative of the position of the head 2a of the applicator 2. Thus, the tracking means/system may provide a signal 6a which provides the position of the head 2a of the applicator 2 with respect to the target object 1.

Particularly, said elongated flexible means/device 3 may be a catheter or a guide wire of a catheter, which guide wire may be preferably configured for transcatheter arterial chemoembolization (TACE). This allows one to combine the creation of an ablation volume 20 with a TACE treatment in an advantageous manner.

The energy control unit 10 is now configured to trigger emission of energy E via the head 2a towards the target object 1 in order to generate an ablation volume 20 comprising the target object 1, once the distance between the head 2a and the target object 1 falls below a certain threshold.

Using the signal 6a, the head 2a of the applicator 2 may also be automatically moved towards the target object 1 along a pre-definable track by means of a means 2d for automatically moving the applicator (such as a robot arm). This also allows one to create an ablation volume 20 of a more complex shape by generating smaller ablation volumes Vi at distinct locations as shown in FIG. 3. However, the applicator 2 may also be guided manually using e.g. a handle 2c of the applicator 2.

In order to be able to visually control and/or observe the ablation process, the tracking means 6 preferably comprises a display means 11 (e.g. a display for displaying information optically) for displaying e.g. the pose of the head 2a of the applicator 2 with respect to the pose of the target object 1.

Further, the complex ablation volume 20 shown in FIG. 3 may also be generated by using several applicators 2, wherein each applicator 2 comprises a head 2a and is configured to be inserted into the patient's body so as to position the respective head 2a into or close to said target object 1. Here, each applicator 2 may be configured to generate an ablation volume Vi which together form the ablation volume 20 shown in FIG. 3.

According to FIGS. 4 to 6, the system according to the present invention can comprise a head 2a that comprises a plurality of independent energy emitting elements Aₙ or A_{n,m} for delivering energy Eᵢ or E_{i,j} to the target object 1 (not indicated in FIGS. 4 to 6), wherein said elements Aₙ, A_{n,m} are arranged side by side along a longitudinal axis L of the respective head 2a and/or side by side along a periphery U of the respective head 2a, wherein particularly the energy control unit 10 is configured to control the respective energy emitting element Aₙ, A_{n,m} so as to generate a desired spatial energy distribution which can be different for different directions, depending on the control, arrangement and characteristics of the respective element Aₙ, A_{n,m}.

Particularly, as shown in FIG. 4, several such elements A₁, A₂, . . . , Aₙ can be arranged one after the other along a longitudinal axis of the head 2a, wherein each element Aₙ is configured to independently emit energy Eᵢ in a cylindrical symmetrical fashion as indicated in the cross sectional view of FIG. 4 in the lower part of FIG. 4.

Alternatively, as shown in FIG. 5, several such elements A₁, A₂, . . . , Aₙ can be arranged one after the other along the periphery U of the head 2a, wherein each element Aₙ is configured to independently emit energy Eᵢ in a certain direction (or predominantly in a certain direction, e.g. in a radial direction with respect to the head 2a) as indicated in the cross sectional view of FIG. 5 in the lower part of FIG. 5.

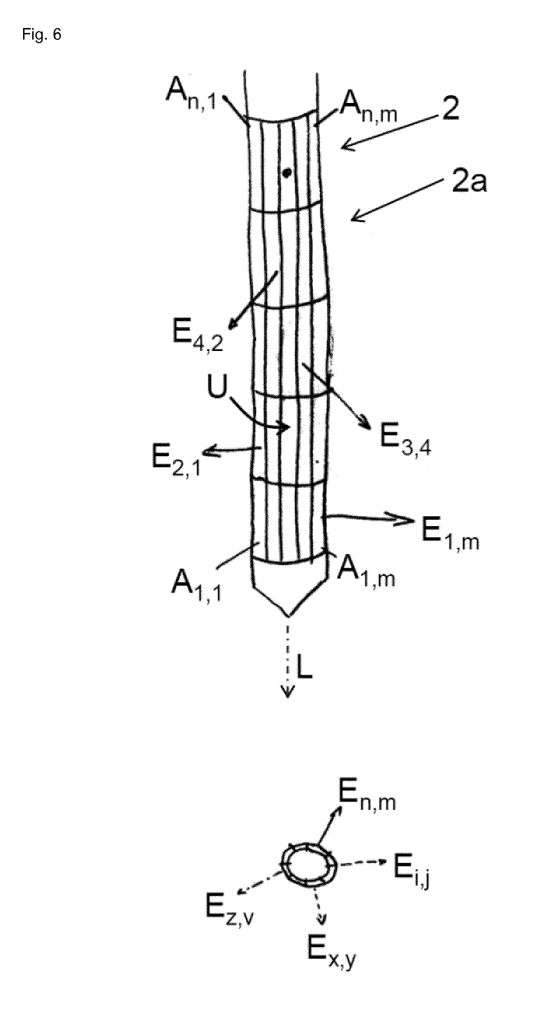

Further, alternatively, as shown in FIG. 6, the embodiments of FIGS. 4 and 5 may be combined, so that the head 2a comprises several such elements A_{n,m} which are arranged along the longitudinal axis L as well as along the periphery U. Thus, the head 2a comprises a two-dimensional array of such elements A_{n,m} arranged on the surface of the head 2a. Here, emission of energy can vary along the longitudinal axis L (as in FIG. 4) as well as in the direction of the periphery U (as in FIG. 5).

Particularly, in an embodiment, the energy control unit 10 is configured to control the individual elements Aₙ or A_{n,m} depending on the relative pose of the head 2a (or of the individual element Aₙ or A_{n,m}) with respect to the target object 1. Thus, energy Eᵢ/E_{i,j} can be delivered to the target object 1 in a very precise manner.

In the following a further aspect of the present invention (item 1) and corresponding embodiments are stated as items, wherein the reference numerals in parenthesis also refer to the drawings. With respect to these items it is also referred to the method described above.

Item 1: A method for controlling the amount of energy (E) emitted per time by a head (2a) of an applicator (2) for creating an ablation volume (20) within a target object (1), wherein data relating to the relative pose of the head (2a) of the applicator (2) with respect to the pose of the target object (1) is measured by means of a tracking system (6) upon guiding the head (2a) of the applicator (2) to the target object (1), and wherein the energy (E) emitted by said head (2a) of the applicator (2) is automatically controlled as a function of said relative pose of the applicator (2) with respect to the target object (1).

Item 2: The method according to item 1, wherein both the pose of applicator's head (2a) and the pose of the target object (1) are measured by means of one of: electromagnetic, optical, optical-fiber-based or time-of-flight based tracking.

Item 3: The method according to item 1 or 2, wherein the temporal and spatial delivery of energy (E) via the head (2a) of the applicator (2) is controlled based on an input (6a) from the tracking system (6).

Item 4: The method according to one of the items 1 to 3, wherein energy (E) is emitted via the head (2a) of the applicator (2) into the target object (1) that causes the tissue in a said ablation volume (20) near or around the target object (1) to be destroyed.

Item 5: The method according to one of the items 1 to 4, wherein the head (2a) of the applicator (2) is guided manually to the target object (1).

Item 6: The method according to one of the items 1 to 4, wherein the head (2a) of the applicator (2) is automatically guided to the target object (1) using a means (2d) for automatically moving the applicator (2).

Item 7: The method according to item 6, wherein said means (2d) is configured to move the head (2a) of the applicator (2) upon emitting energy (E) to the target (1) object in a pre-defined way so as to give the ablation volume (20) a pre-defined shape.

Item 8: The method according to one of the items 1 to 7, wherein the position of the target object (1) is measured using an externally attached tracking device or using an internally placed tracking device (3b).

Item 9: The method according to one of the items 1 to 8, wherein both the pose of the head (2a) of the applicator (2) and the pose of the target object (1) are co-registered to a medical image data set, and wherein, based on the medical image data, anatomical structures are annotated and a simulation and/or extrapolation of the desired outcome is automatically computed.

Item 10: The method according to one of the items 1 to 9, wherein the head (2a) of the applicator (2) provides directionality of energy distribution, and wherein the resulting spatial energy distribution in the target object (1) is controlled as a function of at least one of: the relative position of the head (2) of the applicator (2) and the target object (1), available image data, and additionally available target properties.

Item 11: The method according to one of the items 1 to 10, wherein a plurality of applicators (2) are used that are introduced synchronously or asynchronously into or near the target object (1).

Item 12: The method according to one of the items 1 to 11, wherein energy (E) is emitted into the target object (1) via said plurality of individual applicators (2) so as to create a defined ablation volume (20).

Item 13: The method according to one of the items 1 to 12, wherein the creation of the pre-defined ablation volume (20) is automatically controlled according to a pre-defined image-based plan.

### REFERENCES

[1] Breen, D. J. & Lencioni, R. Nat. Rev. Clin. Oncol. 12, 175-186 (2015); published online 20 Jan. 2015; doi:10.1038/nrclinonc.2014.237

1. A system (S) for creating an ablation volume (20) within a target object (1), comprising:
an applicator (2) comprising a head (2a), which applicator (2) is configured to be inserted into the patient's body so as to position said head (2a) into or close to a target object (1) located inside said body, and wherein the applicator (2) is configured to emit energy (E) provided to the applicator (2) via said head (2a) so as to create an ablation volume (20) comprising at least a region of said target object (1),
a tracking system (6) configured to track the pose (4) of the head (2a) with respect to the pose (5) of the target object (1), wherein the tracking system (6) is further configured to provide a signal (6a) indicative of the relative pose of the head (2a) of the applicator (2) with respect to the target object (1), and
an energy control unit (10) configured to control emission of said energy (E) by the head (2a) of the applicator (2) in response to said signal (6a).

2. The system according to claim 1, wherein the energy control unit (10) is configured to automatically trigger emission of energy (E) via the head (2a) of said applicator (2) towards the target object (1) for generating said ablation volume (20) when the distance between the head (2a) and the target object (1) falls below a pre-defined value.

3. The system according to claim 1, characterized in that the tracking system (6) is configured to determine the pose (4) of the head (2a) of the applicator (2) and the pose of the target object (1) using one of: electromagnetic tracking, optical tracking, optical-fiber based tracking, time-of-flight-based tracking.

4. The system according to claim 1, wherein the tracking means (6) comprises a display means (11) for displaying the pose of the head (2a) of the applicator (2) with respect to the pose of the target object (1).

5. The system according to claim 1, characterized in that the energy control unit (10) is configured to control the temporal and spatial delivery of energy (E) to the target object (1) via the head (2a) of the applicator (2) using said signal (6a).

6. The system according to claim 1, characterized in that the energy control unit (10) is configured to control emission of energy such that the energy that is delivered per unit time and unit of volume to the target object is a linear or non-linear function derived from the relative pose between the head (2a) and the target object (1) or a derivative of said relative pose.

7. The system according to claim 6, characterized in that said delivered energy increases and/or decreases depending on the distance between the head (2a) and the target object (1).

8. The system according to claim 6, characterized in that said delivered energy increases and/or decreases depending on the position of the head (2a) with respect to the target object (1).

9. The system according to claim 6, characterized in that said delivered energy increases and/or decreases depending on the position of the head (2a) and on the spatial orientation of the head (2a) with respect to the target object (1).

10. The system according to claim 1, characterized in that said energy (E) which the applicator (2) is configured to emit via its head (2a) causes the target object (1) to heat up within said ablation volume so that subsequently material properties of the target object (1) in the ablation volume (20) are changed.

11. The system according to claim 1, characterized in that the applicator (2) comprises a handle (2c) for manually moving the applicator (2), or that the system (S) comprises a means (2d) or actuator (2d) for automatically moving the applicator (2).

12. The system according to claim 11, characterized in that the system (S) is configured to generate an ablation volume (20) of a pre-defined shape by moving the head (2a) of the applicator (2) along a pre-defined track by means of said means (2d) or actuator (2d) for automatically moving the applicator (2).

13. The system according to claim 1, characterized in that the tracking system (6) comprises a first tracking device (3b) for measuring the pose (5) of the target object (1), wherein said first tracking device (3b) is configured to be placed into the body of a patient, and wherein preferably the first tracking (3b) device is a first transmitter configured for transmitting an electromagnetic signal, or wherein said first tracking device is an externally attached first tracking device.

14. The system according to claim 1, characterized in that the tracking system (6) comprises a second tracking device (2b) for measuring the pose of the applicator head (2a), wherein said second tracking device (2b) is arranged on the head (2a) of the applicator (2), and wherein preferably the second tracking device (2b) is a second transmitter configured for transmitting an electromagnetic signal.

15. The system according to claim 13, characterized in that the system (S) comprises an elongated flexible means (3) comprising said first tracking device (3b) at a tip (3a) of said flexible means (3), wherein said flexible means (3) is configured to be placed into or close to the target object (1) via a body lumen of the patient.

16. The system according to claim 15, characterized in that said elongated flexible means (3) is a catheter or a guide wire (3) for a catheter.

17. The system according to claim 1, characterized in that the head (2a) of the applicator (2) is configured to provide directionality of energy distribution, wherein particularly the energy control unit (10) is configured to control the resulting spatial energy distribution in the target object (1) as a function of at least one of: the relative pose of the head (2a) of the applicator (2) with respect to the target object (1), the orientation of the head (2a) with respect to the target object (1), the position of the head (2a) with respect to the target object (1), medical image data, a property of the target object (1).

18. The system according to claim 1, characterized in that the head of the applicator (2) comprises a plurality of independent energy emitting elements (Aₙ, A_{n,m}) for delivering energy to the target object (1), wherein said elements (Aₙ, A_{n,m}) are arranged side by side along a longitudinal axis (L) of the head (2a) and/or side by side along a periphery (U) of the head (2a), wherein particularly the energy control unit (10) is configured to control the respective energy emitting element (Aₙ, A_{n,m}) so as to generate said resulting spatial energy distribution.

19. The system according to claim 1, characterized in that the system (S) comprises a plurality of applicators (2), wherein each applicator (2) comprises a head (2a) and is configured to be inserted into the patient's body so as to position the respective head (2a) into or close to said target object (1) located inside said body, and wherein the respective applicator (2) is configured to emit energy (E) provided to the respective applicator (2) via the respective head (2a) so as to create an ablation volume (20, Vi) having a pre-defined shape and comprising at least a region of said target object (20).

20. A method for controlling the amount of energy (E) emitted per time by a head (2a) of an applicator (2) for creating an ablation volume (20) within a target object (1), wherein data relating to the relative pose of the head (2a) of the applicator (2) with respect to the pose of the target object (1) is measured by means of a tracking system (6) upon guiding the head (2a) of the applicator (2) to the target object (1), and wherein the energy (E) emitted by said head (2a) of the applicator (2) is automatically controlled as a function of said relative pose of the applicator (2) with respect to the target object (1).
